(19) United States
(12) Patent Application Publication
(10) Pub. No.: US 2023/0321307 A1
(43) Pub. Date: Oct. 12, 2023

(54) ANTIBACTERIAL/VIRAL DISINFECTING-EFFECTIVE AIR SCRUBBER/PURIFIER FILTER MATERIAL AND FILTERS

(71) Applicant: University of Massachusetts, Boston, MA (US)

(72) Inventors: Yong K. Kim, North Dartmouth, MA (US); Armand F. Lewis, Mattapoisett, MA (US)

(21) Appl. No.: 18/210,724

(22) Filed: Jun. 16, 2023

Related U.S. Application Data
(63) Continuation of application No. PCT/US21/63456, filed on Dec. 15, 2021.
(60) Provisional application No. 63/131,016, filed on Dec. 28, 2020.

(51) Int. Cl. A61L 9/16

(52) U.S. Cl. CPC A61L 9/16

(57) ABSTRACT

An air scrubber/purifying filter includes filter material having a substrate, an adhesive coating applied to the substrate and a plurality of flock fibers flocked into the adhesive coating. In one embodiment, bi-component flock fibers are flocked on a Reticulated Foam (RF) substrate and subsequently fibrillated. In another embodiment, Cross-Flow Flock Fiber (CFF) filter material includes edgewise, cross cut, bonded, stacked fabric layers such that the passage of air through the flock fibers is normal to the flock fiber orientation. In some embodiments the flock fibers are coated with biocidal, virucidal and/or metalized coatings or finishes to provide biocidal or virucidal properties to the filter media.

### RELATED APPLICATIONS

This application is a national stage filing of PCT application No.: PCT/US21/63456 filed Dec. 15, 2021, entitled ANTIBACTRIAL/VIRAL DISINFECTING-EFFECTIVE AIR SCRUBBER/PURIFIER FILTER MATERIAL AND FILTERS, which itself claims priority to U.S. Provisional Patent Application No. 63/131,016 filed Dec. 28, 2020, entitled ANTIBACTERIAL/VIRAL DISINFECTING-EFFECTIVE AIR SCRUBBER/PURIFIER FILTER MATERIAL AND FILTERS, the entire teachings of all of which are incorporated herein by reference.

### FIELD OF THE INVENTION

The invention relates to air scrubbing and purifier filter materials, methods of manufacture and filters made from these materials.

### BACKGROUND

It is generally known that COVID-19 viruses are readily transmitted in places of human occupancy such as hospitals, nursing home rooms, medical care facilities, educational facilities and business offices by air contamination. Many of these locations are not adequately ventilated and during functional human occupancy, it is expected that rampant air contamination could easily characterize these areas. If any one or more persons in a confined access room are virus contaminated, highly contagious viruses can circulate and easily contaminate the air in the room in the form of aerosol particles; colloidal, ultra-small (80 to 160 nanometer) fluid particles stably suspended in air. There are a number of single-room, effective air purifying devices on the market (e.g., Honeywell, Westinghouse, NuWave, Medify MA and Hathaspace). Each of these devices employs a layered filtration media through which the room air is continuously passed (and recirculated). There is a need for suitable filter material to be used in these devices. The use of sea-island bi-component fibers as flock fibers for nasal swabs has been disclosed in U.S. Pat. No. 8,334,134, which is hereby incorporated by reference herein in its entirety.

High-Efficiency Particulate Absorbing (HEPA) filters, also known as high-efficiency particulate absorbing and high-efficiency particulate capture, is an efficiency standard for air filtration. Filters meeting the HEPA standard must satisfy certain levels of efficiency. Common standards require that a HEPA air filter must remove from the air that passes through at least 99.95% (European Standard) or 99.97% (ASME, U.S. of particles whose diameter is equal to 0.3 μm; with the filtration efficiency increasing for particle diameters both less than and greater than 0.3 μm. (DOE Standard Specification for HEPA Filters; Section 5.2.1 Penetration: Aerosol penetration for any HEPA filter shall not exceed 0.03% (0.0003) at 0.3 micrometer particle size).

HEPA was commercialized in the 1950s, and the original term became a registered trademark and later a generic term for highly efficient filters. HEPA filters are used in applications that require contamination control, such as the manufacturing of disk drives, medical devices, semiconductors, nuclear, food and pharmaceutical products, as well as in hospitals homes and transportation vehicles. Many types of HEPA filter materials are available commercially. There are also many classifications of HEPA filter systems which are based on their specific ability to filter out various particle sizes from the air. A typical configuration of a conventional HEPA filter 10 includes a filter frame 12 housing continuous sheets of nonwoven filter media 14 packed between corrugated aluminum separators 16 to increase the filtration surface area as shown in FIG. 1. It is noted in that these air filtration media are basically planar material sheets, fabricated into a corrugated form to increase their surface area per areal geometric shape of the filter media. The path through which the air passes is not rendered tortuous by this corrugated sheet arrangement. While this configuration may be most effective in filtering out particulate matter from air, this structure is not conducive to the disinfecting of air as the air passes through the filter; the air does not pass through a complex path. In such single layer, corrugated sheet HEPA filter material configurations, the residence time of the passing-through air is very short.

### SUMMARY

The air scrubber/purification media material disclosed herein differ significantly from conventional HEPA filter material. Embodiments disclosed herein feature a filter structure that has a through-thickness complexity. Because of the presence of the flock fibers, the air (or vaporous liquid) is made to pass through a high surface area, thicker cross-section, convoluted, tortuous path where the air-to-solid surface material media contact is maximized. Such an arrangement is ultimately conducive to imparting air disinfecting/purification effects to an air scrubber's overall effectiveness.

Highly contagious bacterial and viral agents contaminate the air in the form of aerosol particles; colloidal, ultra-small (80 to 160 nanometer) fluid particles stably suspended in air. From this premise, one methodology of reducing the spread of particulate airborne diseases in human habitation areas is by a ubiquitous use of effective air cleaning, individual room and personal air-scrubbing/purifying respirator air cleaner/purification filter systems.

Filtration materials concepts employing the special treatment of flocked fiber surfaces are disclosed herein. Ultra-Effective COVID-19 destroying, air scrubber media materials and other airborne biohazard particle bio-neutralizing aerosol particle absorbing filtration media are described below. These materials and technologies include: (a) the use of silver, copper or copper alloy metal coated flock fiber fibrous material filter layer assemblies; silver, copper and their alloys metal surfaces have excellent antibacterial/antiviral properties, (b) post-fibrillated bi-component flocked fibers can be used to produce very high surface area, absorbent air filter media, and (c) flocked, open-cell reticulated foam material structures have shown promise as the base material for creating low-pressure drop air filtration structures. Combining air-scrubber/purifying filtration systems with in-line Light Emitting Diode (LED) created ultra-violet (UV) light also serves as an effective air disinfecting methodology.

It is well recognized that many single-room, human habitation areas are characterized by poor to no outside air circulation. Therefore, air in these confined areas can easily become contaminated by unwanted human-borne pathogens. Devices and material disclosed herein address this problem. Two air scrubbers/filter materials are disclosed below: (1) Flocked Reticulated Foam (FRF) air filtration material and (2) Cross-Flow Flock Fiber (CFF) ply-layered filter media. These filtration media are based on using flock fiber to impart high surface area, low back-pressure air-filtration features to special filtration media structures. Furthermore, by treating these flock fiber media with suitable disinfecting biocides, germicides or virucides, in a proper arrangement, these filtration structures are able to continuously and effectively capture and destroy airborne hazardous particles and purify the room air. These treatments can be applied, for example, by dipping and spraying followed by suitable drying and/or curing processes. These air scrubber/filtration media can be used as retrofit air-flow through panels in suitable unitary air purifying devices. These so-modified unitary air purifiers can then be readily deployed in single-room educational, business, nursing home and health care facility and otherwise community-room habitation areas to continually sanitize the room air.

In one embodiment, an air scrubber/purifying filter includes flocked media which include: adhesively coated substrate, a plurality of flock fibers flocked into the adhesively coated substrate and the plurality of flock fibers comprises air disinfecting flock fibers. In a further embodiment, the flock fibers include at least one of: a pass-through airborne pathogen destroying biocidal coating; a pass-through airborne pathogen destroying virucidal coating; a metalized coating; a pass-over airborne pathogen destroying biocidal coating; a pass-over airborne pathogen destroying virucidal coating; a metal oxide coating; and a metalized coating.

In another embodiment, the air scrubbing/purifying filter substrate includes a fabric substrate and the filter further includes a plurality of layers of flocked media. The plurality of flocked fabric layers is arranged such that airflow through a filter cross-section passes through the plurality of flock fibers in a direction normal to the upright orientation of the flock fibers. These multiple flocked fabric ply layers are secured together by mechanical entanglement action or else bonded together on top of each other such that controlled thickness sections of the flocked fabric are so arranged that airflow through the assembly of flocked fabric layers passes through the flock fibers in a direction normal to the upright orientation of the flock fibers. Such a Cross-Flow Flocked (CFF) filter material configuration provides a very high contact surface area air-pass-through fibrous media for use in air scrubbing/purifying filtration systems.

In another embodiment, the adhesively coated substrate includes a reticulated foam substrate; and the plurality of the air disinfecting flock fibers include germicide/biocide/virucide treated flock fibers. This embodiment involves flocking flock fiber onto open-cell Reticulated Foam (RF). Flocked Reticulated Foam (FRF) is a versatile filter media configuration capable of successfully operating at low back pressure and can be designed to have high air retention times. The flock fibers of these FRF structures are also readily adaptable to the application of germicidal/biocidal/virucidal coatings to flock fibers giving these FRF structures excellent air disinfecting properties. In a still further embodiment, the air scrubbing/purifying filter substrate includes a reticulated foam substrate and the plurality of flock fibers includes fibrillated bi-component fibers. The plurality of fibrillated bi-component fibers can include biocide/virucide treated fibrillated bi-component flock fibers. In these embodiments, the bi-component flock fibers are fibrillated to form an Ultra High Surface Area flocked FRF media structure and the flock fibers include disinfecting properties. Such an arrangement curbs the spread of unwanted, airborne, human carried hazardous pathogens such as COVID-19.

A method of making an air scrubber/purifying filter includes providing a substrate; providing flock fibers having disinfecting properties; applying adhesive to the substrate; and flocking the flock fibers having disinfecting properties onto the substrate to form a flocked fiber layer. In a further embodiment, the method includes coating the flock fibers with at least one of: a biocidal coating; a virucidal coating; a pass-through airborne pathogen destroying biocidal coating; a pass-through airborne pathogen destroying virucidal coating; a pass-over airborne pathogen destroying biocidal coating; a pass-over airborne pathogen destroying virucidal coating and a metalized coating.

In another embodiment, the air scrubber/purifying filter is a stacked assembly of Cross-Flow Flocked fabric filter layers; and the method further includes the steps of: stacking a plurality of flocked fabric layers; bonding the plurality of stacked, flocked fiber layers together on top of each other; edgewise cross-cutting the bonded plurality of cross cut, stacked, flocked fiber layers; and coating the flock fibers with one of: an airborne pathogen destroying biocidal coating; an airborne pathogen destroying virucidal; a metalized coating; a pathogen contaminated air pass-over biocidal coating; a pathogen contaminated air pass-over virucidal coating; a metalized coating and a metal oxide coating. In yet another embodiment, the substrate comprises a porous reticulated foam (RF) substrate and the method further comprises applying a vacuum to the porous RF structure while flocking the flock fibers onto the porous RF substrate.

In response to the problems described above, embodiments disclosed herein, use a unitary pathogen destroying air purification device. Such devices serve to continually recirculate the air in the room rendering it pathogen-free for human occupancy. Embodiments as disclosed herein employ high surface area fiber flocked surfaces as the filtration media described above. This is in contrast to increasing the surface area of the filter by the presently used folded, corrugated, thin, simple air passage, lower surface area bulk-thickness increasing puckered flat sheet HEPA filter material and configurations. In contrast to conventional material, the fibrous, high surface area media disclosed herein are designed to make the air pass through a much more convoluted, tortuous pass where the residence time of the air (gas/solid surface) interface is much higher that what is possible with existing HEPA type filter media. It is noted also that these features of the flocked filtration media are accomplished with the minimum of air-flow back pressure. Additionally, the air residence time factor for this filter material is readily controlled and designed into the media material, choosing various flock fiber denier and length variations. The surface of this flock fiber enhanced, fibrous media is also easily modified by the surface application of disinfecting treatments such as copper oxide nano-particles, silver metal. silver nano-particle and/or Micro-Ban® like biochemical agents. Embodiments disclosed herein, include, but are not limited to having the following features:

(1) Very high surface area flocked surfaces to enhance air/solid surface contact effectiveness;
(2) Flock fiber denier, length and flock density that can control the air-flow back pressure effect to minimal limits;
(3) Copper oxide, silver metal/silver nano-particle coated disinfecting surfaces with high surface area that will enhance the desired disinfecting effect;
(4) Very broad air filter design and shape capabilities;
(5) Final configurations having very good retro-fit adaptability to existing unitary air filter systems and/or room air purifying devices/installations; and
(6) Compatibility with LED powered UV radiation virus killing treatment.

As used herein, the term airborne generally refers to the status of unwanted suspended in air pathogen particles or vapors. Airborne refers to the place where the virulent disease-causing pathogens are located (e.g., in the air (gas phase); stably suspended in air). Pass-over refers to the passing of the contaminated air over and past the surface of the media material; Passing through the media refers to the contaminated air passing through the cross-sectional, media thickness (i.e., passing through such as through a sieve or strainer or layer of filtration media). While the contaminated air is Passing Through the media's cross-section, it will also Pass Over the germicide/biocide/virucide treated flock fibers and through the thickness of the media material. In an air scrubbing/purifying process the contaminated air passes through the media and along the way it passes over the biocide/virucide coated flock fibers.

### BRIEF DESCRIPTION OF THE DRAWINGS

The foregoing and other objects, features and advantages of the invention will be apparent from the following more particular description of embodiments of the invention, as illustrated in the accompanying drawings and figures in which like reference characters refer to the same parts throughout the different views. The drawings are not necessarily to scale, with emphasis instead being placed upon illustrating the embodiments, principles and concepts of the invention. These and other features of the invention will be understood from the description and claims herein, taken together with the drawings of illustrative embodiments, wherein:

FIG. 1 is a schematic diagram of a conventional HEPA filter;

FIG. 2A is a diagram of an assembly of stacked layers of fiber flocked fabric that have been assembled together in accordance with one example embodiment disclosed herein;

FIG. 2B is a diagram of a cross-sectional slice of an assembled stacked layer of fiber flocked fabric of FIG. 2A;

FIGS. 3A-3D are diagrams of air passage through filtration media mechanism in accordance with example embodiments disclosed herein;

FIG. 4A is a photograph of open cell Reticulated Foam (FRF) in accordance with an exemplary embodiment disclosed herein; and

FIG. 4B is a photograph of flocked reticulated polyurethane foam in accordance with an exemplary embodiment;

FIG. 5A is a photograph of Adhesive Coated Reticulated Foam (FRF) in accordance with an exemplary embodiment disclosed herein; and

FIG. 5B is a photograph of flocked reticulated polyurethane foam in accordance with an exemplary embodiment; and

FIG. 6 is a diagram of a cross-sectional illustration of a Sea-Island component fiber in accordance with one example embodiment disclosed herein.

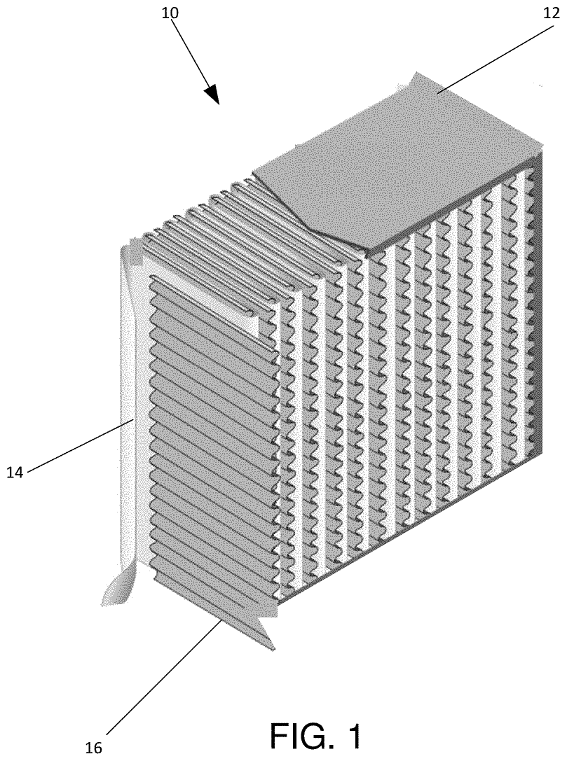

### DETAILED DESCRIPTION

Air scrubber type filters are critical in the prevention of the spread of airborne bacterial and viral organisms and, therefore, infection by these organisms. Typically, medical use HEPA filtration systems also incorporate high-energy ultra-violet light units or panels with anti-microbial coating to kill the live bacteria and viruses passing through and trapped by the filter media. Some of the best-rated HEPA units have particle capture efficiency ratings of 99.995%, which assures a very high level of protection against airborne, particulate, disease transmitting agents such as COVID-19 viral aerosol. To reduce size to fit a self-contained, powered, air-purifying respirator (PAPR) fitted with an air scrubber filter for medical staff or patient isolation chambers used to protect medical personnel from airborne or aerosolized pathogens such as COVID-19 virus, requires changes in filter media design and configuration.

Two different COVID-19 aerosol virus filter media configurations are described below. Both of these concepts employ flocked fiber surfaces as an important feature. Flocked surfaces are a very effective way of increasing the air/media contact area in these air scrubber filtration media systems. This air/media surface area increase methodology is much more effective than the conventional folded or corrugated-sheet filter media surface area increasing methodology.

### Cross-Sectional Flow Flocked (CFF) Antimicrobial Flock Fiber Surfaces

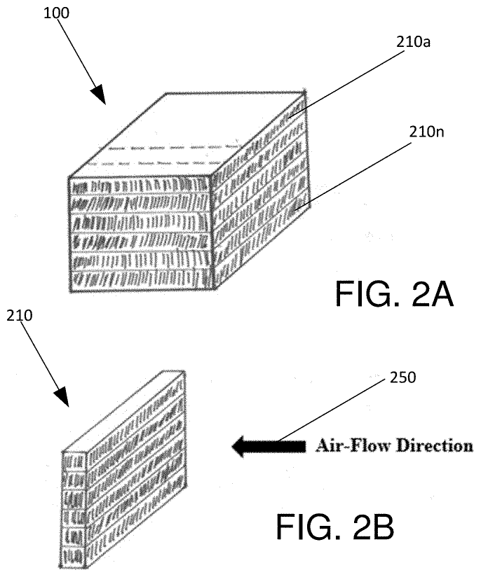

Now referring to FIGS. 2A and 2B, diagrams of an ultra-high surface area, Cross-Flow Flocked (CFF) layer filter 100 and an individual CFF flocked fabric layer 210 are shown. In one embodiment, Cross-Flow Flocked (CFF) layer filter 100 includes multiple flocked fabric layers 210a-210n (generally referred to as flocked fabric layer 210). FIG. 2B shows the air flow direction indicated by arrow 250 though the flocked fabric layer 210. Various embodiments include flocked fiber surfaces coated with antimicrobial functional finish including copper oxide, silver, copper, or copper alloy ions and/or metals form layers 210 which are stacked and bonded together to form a very thick layered structure 100. These stacked flocked layers are then edge-sliced into desired thickness cross-sectional layers. In thinner cross-sectional cross-flow flocked media designs, the stacked-up fiber flocked layers are adhesively bonded together to maintain the thin cross-section dimensional stability of the CFF. These CFF embodiments result in a fibrous media material where the passage of air is normal to the upright direction for the aligned flocked fibers. Air flow will occur through the side of the upright flock fiber mass as indicated by arrow 250 Air-Flow Direction in FIG. 2B. The air scrubber filter media in these embodiments are referred to as the Cross-Flow Flocked (CFF) media.

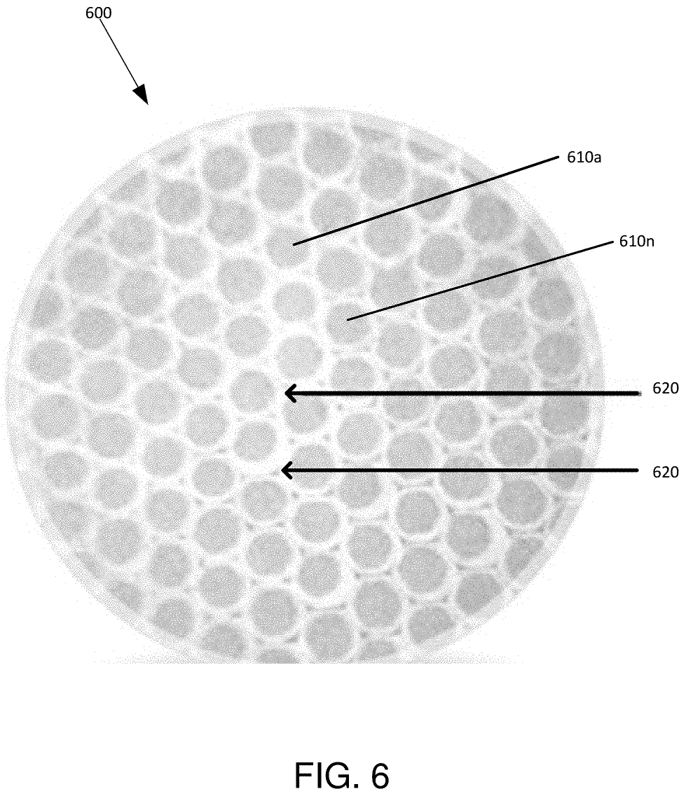

In certain embodiments, CFF media are fabricated with regular flock fibers (e.g., 1-40 denier, 1-6 mm length) and/or fibrillated bi-component fibers as the flock fibers. Bi-component fibers substantially increase the surface area of the filter media (FIG. 6 below shows further details of bi-component fibers). Bi-component fibers are chemically treated to produce very high surface area, nano-fibrous, micro-fiber particle capturing fibrous structures.

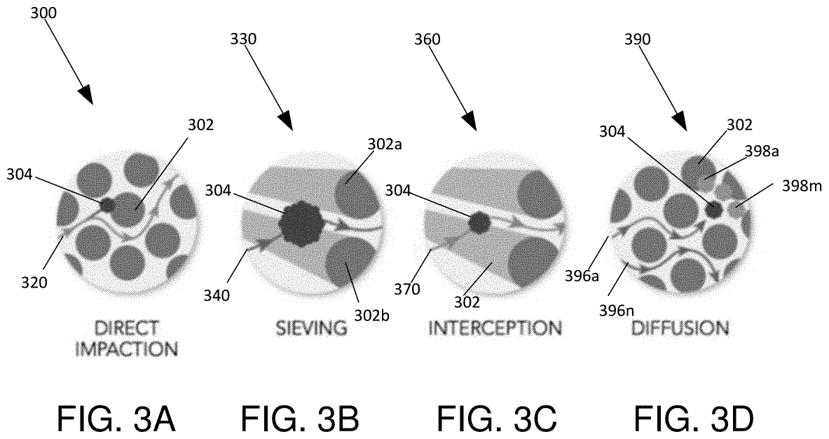

In one embodiment having thinner cross-section CFF media configurations equal to or thinner than about four (4) inches, the individual and stack assembled, flock coated fabric layers are bonded together. Tops of flock fiber surfaces are adhesively bonded to the bottoms of the adjacent (contiguous) flocked fabric layer (i.e., both ends of the flock fibers are bonded to a fabric). Adhesives include, but are not limited to water based acrylic and polyurethane based adhesives. Additionally, antibacterial/virucidal chemically modified flock adhesives can be used. Optimized CFF media include metalized or regular flock fiber (nylon, polyester, viscose, or acetate fibers, 1 to 60 denier 0.5 to 10 mm long), and/or post fibrillated bi-component flock fiber (nylon, polyester fibers, 1 to 60 denier 0.5 to 10 mm long, 16-36 islands). In another embodiment the fiber length is about 5 mm. These media can meet the air scrubber and antiviral function desired for medical staff and patient isolation chamber applications. To capture finer particle less than one micrometer, an electret filter media layer can be sandwiched in between CFF elements. Generally, filtration mechanisms involve the passage of air through convoluted and tortuous paths such as: impaction, sieving, interception and diffusion as shown in FIGS. 3A-3D. FIG. 3A shows direct impaction 300 of particle 304 into fiber 302 along path 320. FIG. 3B shows sieving 330 of particle 304 by fiber 302a and 302b along path 340. FIG. 3C shows interception 360 of particle 304 by fiber 302 along path 370. FIG. 3D shows diffusion 330 of particle 304 along paths 396a-396n. Particles 398a-298m are diffused dirt particles entrapped in the filter media.

In CFF configurations, the surface area created by flocking in certain embodiments is about 40-fold greater than the substrate area of a flat/folded sheet filter material. Thus, the effective CFF filtration surface area is proportional to the number of the stacks, the flock fiber cross sectional area and the flock density. This relatively large media surface area effectively interacts with particles by increasing path length and interacting surface area for arresting (capturing) particles by the direct impaction, sieving, interception, diffusion and surface pass-over. The antimicrobial textile fiber surface finishes and/or the silver, copper or copper alloy metallized flocked layers create an extraordinarily effective air disinfecting effect on the incoming ‘flow-through’ contaminated air. Air disinfecting occurs when airborne pathogens contact or rub against the biocide/virucide coated flock fiber material. Full particle capture and retention by the media, while helpful, it is not necessary for the biochemical destruction of the flow through airborne bacteria or viruses.

After flocking, the layers of flocked fabric are stacked, bonded and cross cut (cut edge wise) and configured such that controlled thickness sections of the flocked fabric is so arranged that airflow through the cross-section of the CFF media passes through the flock fibers in a direction NORMAL to the upright orientation of the flock fibers. This is unique and serves to produce very high contact surface area air-pass-through fibrous media material systems.

The disinfecting coating can be applied to the flock fibers The coating, includes but is not limited to, a metalized coating; an airborne biocidal coating; an airborne virucidal; airborne pass-over effective biocidal coating; an airborne pass-over effective virucidal coating; a metal oxide coating; and combinations thereof. The coatings are generally applied after the flocking process, but can be applied before flocking. The coatings can be sprayed on or applied by dipping.

### Flocked Reticulated Foam (FRF)

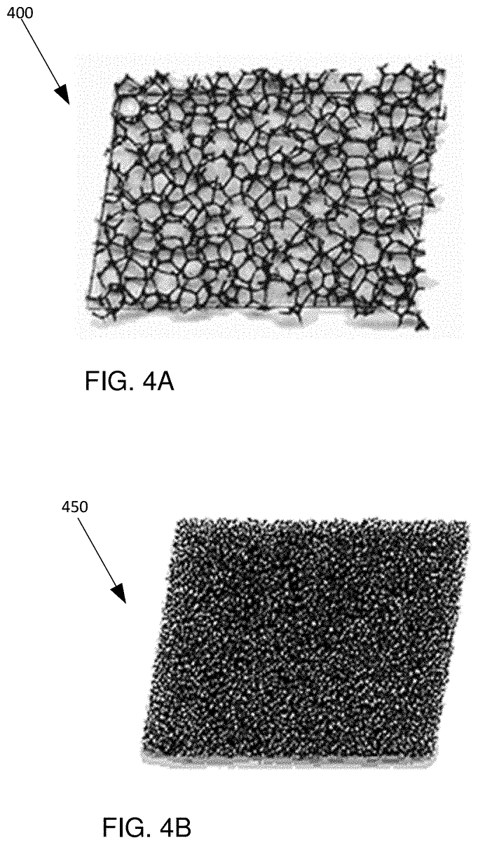

Reticulated foams are a special form of very porous, open cell foams that are used for many applications such as in air and liquid filtration media, packaging, sound absorption, wiping pads and the like. Reticulated foam materials are different from typical foams in that their pore cells are open unlike the closed cell (encapsulated bubble) air inclusions that characterize ordinary foam materials. Reticulated foams are produced by a two-step process that begins with (1) conventional (closed cell) polyurethane foam and (2) removing the cell faces (polymeric membranes between the formed air “bubble” inclusions) that convert the foam material from a closed-cell foam to an open-cell foam. Converting the closed cell foam to reticulated (open-cell) foam can be done by thermal “zapping” or chemical “quenching” processes. In the “zapping” process, the closed-cell foam is placed in a pressurized container into which a pressurized gas (e.g., hydrogen) is introduced. This combination then undergoes a “controlled explosion” which destroys the closed cell nature of the conventional foam forming an open-cell. A “netting-like” material structure is thus formed known as reticulated foam. FIGS. 4A-4B illustrates two types of open cell reticulated foam having different porosities. Overall, reticulated foam is produced in a porosity range of from 1 to 100 pores per sq. inch (ppi). In another embodiment the range is about 1 to 50 ppi. Both polyester and polyether based polyurethane foams can be used. FIG. 4A shows a very open cell reticulated foam having about 10 pores per sq. inch and FIG. 4B show a reticulated foam having about 45 pores per sq. inch.

Adding the flock fibers to the reticulated foam structures serves to increase air contact surface area to the already high surface area reticulated foam structures. Adding flock fiber is generally applicable to the higher porosity reticulated foams, for example, the 8 to 45 pores per sq. inch (ppi) cell-size range. While smaller cell size reticulated foam can be flocked, with small cell size reticulated foams, there is no assurance that the small cell pore sizes will not be blocked or sealed up by the needed flock adhesive that must be applied to accomplish the flocking process.

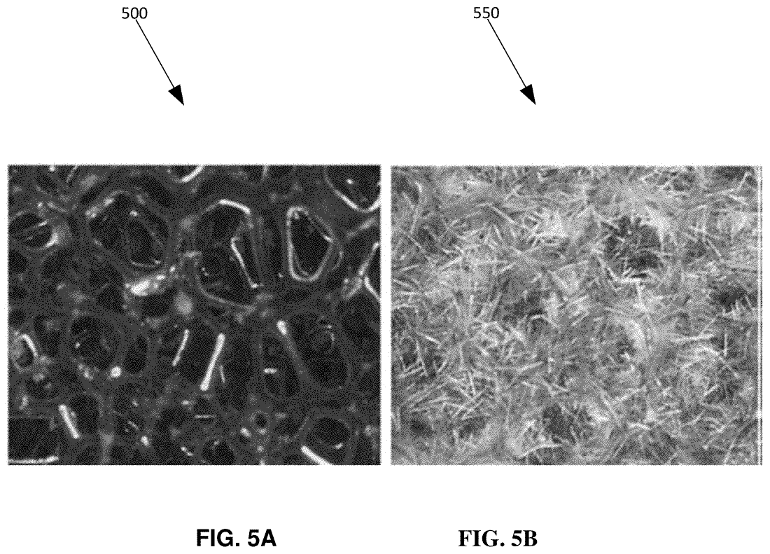

When fabricating Flocked Reticulated Foam (FRF) several features of the flock fibers are taken into consideration for example: (a) the flocked fibers must be of low denier and (b) the flock fibers must be no longer than the average diameter of the geometric pores of the Reticulated Foam. The feature of using low denier flock fibers addresses the need to produce FRF structures having low back pressure. Here, with having higher flexibility, low denier (e.g., 1-10 denier) flock fibers in the pores of the FRF, these flexible flock fibers can easily bend or move in the direction of the air flow. This flock fiber bending reduces the back pressure effect in these FRF filtration structures. In the Flocked Reticulated Foam (FRF) embodiments, it is the bending of the thin cross-section flock fibers (low denier or fibrillated) in the flow direction of the pass-through air that enables reduced back pressure. These thin, low diameter flock fibers are able to move with the flow while still in line to capture and disinfect the passing pathogen particles. Diagrams of the FRF structures studied are presented in FIGS. 5A and 5B.

FIG. 5A shows an adhesive coated reticulated foam (FRF) control with hole sizes of 4±2 mm and FIG. 5B shows a flocked reticulated polyurethane foam (magnified 20× by Olympus SD-STB3 Video Microscope; here 1.2 mm long. 2.4 denier polyester flock fibers are shown).

Table 1 below summarizes data which are the results of back pressure experiments. In these tests, foam samples like those shown in FIG. 5A and 5B of the type Reticulated Foam (RF) RF-9 (this type of RF has a 9 pores/sq. in number of holes) which were placed over the 10-inch×10-inch vacuum port of a Down Flocking, flocking booth. Results obtained show that some degree of air flow restriction was present when RF and FRF sample material were placed over the vacuum port. All results were compared to the OPEN (no sample) Vacuum Table port air flow rate of 3.6 m/s. The Table 1 data show that the back pressure of a tested RF-9 panel increases with foam panel thicknesses between ½″ and 1.″ As the panel thickness is increased to 1½″, the back-pressure effect is only slightly less. Also, adhesive resin coated onto the RF does not have any measurable effect on the foam's back-pressure performance.

TABLE 1
Flocked Reticulated Foam Filter Media Materials Air Flow Data

| | | | Flock | Area | Forced |
|---|---|---|---|---|---|
| | | Type of | Dimensions | Increase^{d} | Air Flow |
| | Thickness | Flock | Length/ | (square | Rate^{c} |
| RF Type^{a,b} | (inches) | Fiber | Denier | meter) | (m/s) |
| OPEN VACUUM TABLE 10″ × 10″ HOLE - -No Sample | | | | | 3.6 |
| RF-9-S | ½″ | PET | 0.048″/2.4 | 0.38 | 2.5 |
| RF-9-S | 1″ | PET | 0.048″/2.4 | 1.11 | 2.0 |
| RF-9-VA | 1½″ | PET | 0.048″/2.4 | 1.23 | 2.1 |
| RF-9-VA | 1″ | Nylon | 0.150″/15 | 0.69 | 2.1 |

CONTROL SAMPLES - - NOT FLOCKED

| | | | | | |
|---|---|---|---|---|---|
| RF-9 AR | ½″ | NA | NA | NA | 2.7 |
| RF-9 RC | ½″ | NA | NA | NA | 2.7 |
| RF-9 AR1 | 1″ | NA | NA | NA | 2.2 |
| RF-9 RC2 | 1″ | NA | NA | NA | 2.2 |
| RF-9 AR | 1½″ | NA | NA | NA | 2.3 |
| RF-9 RC | 1½″ | NA | NA | NA | 2.5 |
| RF-9 RC1 | 1″ | NA | NA | NA | 2.3 |
| RF-9 RC2 | 1″ | NA | NA | NA | 2.2 |

^{a}RF-9 reticulated foam has a 9 pores per square inch pore size - -equates to a hole size of 0.16″ +/− 0.08″ dia. S = Standard flocking; VA = Vacuum Assist;
^{b}RF-9 AR refers to As Received (not flocked) foam; RF-9 RC refers to Resin Coated foam which is immersed into an adhesive bath, the excess adhesive is squeezed out and the adhesive covered foam is allowed to dry at room temperature;
^{c}Represents the rate of air flow through the RF sample when placed over a 10″ × 10″ square (flat) air flow port in UMD's Down-Flocking processing booth. A vacuum is imposed on this 10″ × 10″ hole in the “bed” of the flocking booth when the blower fan of the booth is turned on; and
^{d}Area Increase in the table heading is determined by the weight of the flock fiber measured for each flocked panel. Calculated from the weight of the flock and its length and denier, the increase is surface area was estimated.

FRF panels listed in Table 1 exhibit a slightly higher back-pressure than the non-flocked panels. As expected, low denier flock fiber works well in this application since the RF surface/interior pore attached flock fibers are able to flex or bend in the direction of air flow. This flock fiber bendability effect should serve to lower the imposed back-pressure effect. Notably, by adding flock fibers to the base open cell reticulated foam increases the surface area of the material and the interior pore complexity of the filter.

When fabricating FRF media structures for air-scrubber/purifying applications, it is beneficial to add as much flock fiber to the base Reticulated Foam structure as feasible; more flock fiber means higher surface area which leads to better air filtration/treatment effectiveness. To this end, it was found that if a vacuum is applied to the porous RF structure while the flocking process is in progress, the applied flock fibers are more apt to penetrate into the thickness (cross section) of the RF structure. In this process, the adhesive coated Reticulated Foam (RF) is flocked while a vacuum is steadily applied to the porous foam structure. In one embodiment a Down Flocking process is used. It is noted that with this process the flock fibers are “sucked” down to enter more deeply into the porous RF structure. Vacuum assisted flocking causes the flock fiber to more deeply penetrate the RF cross-sectional thickness and increases the amount of flock fibers deposited and consequently renders higher surface area into a given thickness of RF.

It is noted that there might be a limit to the thickness of individual FRF air scrubber/purifying media material layers. FRF thicknesses of under 1″ thick would be the preferred thickness, say thickness under ¾.″ In all cases, it also is most effective if flock fiber is added to each side of the Reticulated Foam for best results. It is found that flock fiber penetration depths depend upon the porosity level of the RF. The larger the pores the deeper the flock will penetrate the structure of the RF, including of course vacuum assisted flocking. Considering now a nine ppi RF it is found that flock penetration depths of about 3/16″ are achievable, therefore a total FRF thickness of about ⅜″ (with two sides flocked) would be the optimum thickness for a FRF structure fabricated using approximately nine ppi porosity RF.

In other embodiments of this FRF air-scrubber/purifying material system, disinfecting coatings are generally and easily applied after the flocked Reticulated Foam or Flocked Fabric has been fully fabricated (i.e., flock fibers are fully cured in place upon the substrate). It is noted that if the biocidal and/or virucidal coating is on the flock fiber before flocking, the coating could interfere with the adhesion of the flock fiber to the substrate. Also, the final curing of the flock adhesive may interfere with the effectiveness of the biocidal/virucidal coating; heat of curing may destroy the coatings disinfecting effect. If any organic chemically based disinfecting coating is applied to the flocked media it should generally be applied after flocking. For metallized vapor (“inorganic”, metal oxide) type disinfectant coatings, these coatings can be applied onto the Flock Fibers before the flocking of the substrate. These types disinfecting coatings/treatments should be quite stable at high flock adhesive curing temperatures. Also, these inorganic disinfecting coatings may not be as detrimental to the adhesion of the flock fiber to the substrate. In one embodiment the disinfecting coatings (e.g., organic chemical biocides/virucides and inorganic and metallic or metallic oxide flock fiber disinfecting coating) are applied to the air scrubber/purifying media materials.

Fibrillated Bi-Component Fibers Flocked Deposited onto Reticulated Foam

Now referring to FIG. 6, a Sea-Island Bi-component fiber 600 includes “Islands” 610a-610n and the Sea 620 a continuous phase surrounding the “Islands” 610a-610n. In one embodiment, a filter includes FRF filter media which use fibrillate-able bi-component fibers as the flock fibers. This embodiment substantially increases the surface area of the filter media. Bi-component fibers are chemically fibrillated to produce very high surface area, nano-fibrous, micro-fiber particle capturing fibrous structures. FIG. 6 shows a cross-sectional depiction of Sea/Island type bi-component flock fibers, here, polymer fibers (e.g., polyester fibers, 1 to 5 denier 0.5 to 10 mm long and including 16-36 islands in sea). Fibers are flocked onto and into the open structure of reticulated (polyurethane) foam (see FIGS. 5A and 5B). These flock fibers are then be fibrillated by treating the bi-component flock fiber fabric layer with 5% sodium hydroxide (alkali) water solution. This process dissolves out the “sea”, continuous polymer component of the fiber leaving or exposing the insoluble-to-alkali very small nanosized “island” component of the original bi-component flocked fiber. The resulting structure is an ultra-high surface area flocked surface having enhanced air scrubber/purifying properties. In an additional step, a germicidal/biocidal/virucidal coating is applied to the fibrillated flock fiber surface. Microban® or other biochemically active agents such as copper oxide, silver metal vapor deposited chemical anti-pathogen treatments can be applied. The extrudable/fibrillatable polymers fibers can include, but are not limited to, Polyester/Polyester Nylon/Polyester, Polypropylene/Polyester fibers.

Biocidal Effectiveness Testing Procedure per American Association of Textile Chemists & Colorists (AATCC): Using an appropriate spray device, a very light coat (fine mist) of disinfecting biocides or virucides is applied to sterile glass Petri dish carriers (100 mm in diameter) and allowed to dry under laminar flow conditions from 1 hour to overnight.

SARS-CoV-2 pseudo-virus suspensions are thawed and the control and coated carriers are inoculated with a volume of virus suspension containing an adequate titer to recover a minimum of 4-Log¹⁰ infectious viruses per carrier.

Inoculated carriers are held for the predetermined contact times (e.g., 0, 15, 30, 60, minutes). Virus is then recovered from both test and control carriers by addition of an appropriate volume of neutralizing buffer or PBS followed by gel filtration.

Following neutralization of test and control carriers, the viral suspensions are quantified to determine the levels of infectious virus using standard cell culture techniques.

Recovered virus is used to spin-infect human small airway epithelial cells (HSAECs), or Beas 2B cells in a 12-well plate (931 g for 2 hours at 30° C. in the presence of 8 ug/ml polybrene). Fluorescence microscopic images were taken 18 h after infection. Flow cytometry analysis of ZsGreen+ cells are carried out 48 hours after infection by flow cytometry and with the FlowJo software.

The appropriate calculations are performed (e.g., Spearman-Karber) to determine viral titers.

Log¹⁰ and percent reductions are computed for virus exposed to the biocide/virucide product relative to the titer obtained for control carrier(s).

All literature and similar material cited in this application, including, patents, patent applications, articles, books, treatises, dissertations and web pages, regardless of the format of such literature and similar materials, are expressly incorporated by reference in their entirety. In the event that one or more of the incorporated literature and similar materials differs from or contradicts this application, including defined terms, term usage, described techniques, or the like, this application controls.

The section headings used herein are for organizational purposes only and are not to be construed as limiting the subject matter described in any way. While the present invention has been described in conjunction with various embodiments and examples, it is not intended that the present teachings be limited to such embodiments or examples. On the contrary, the present invention encompasses various alternatives, modifications, and equivalents, as will be appreciated by those of skill in the art. While the teachings have been particularly shown and described with reference to specific illustrative embodiments, it should be understood that various changes in form and detail may be made without departing from the spirit and scope of the teachings. Therefore, all embodiments that come within the scope and spirit of the teachings, and equivalents thereto are claimed. The descriptions and diagrams of the methods of the present teachings should not be read as limited to the described order of elements unless stated to that effect.

The claims should not be read as limited to the described order or elements unless stated to that effect. It should be understood that various changes in form and detail may be made without departing from the scope of the appended claims. Therefore, all embodiments that come within the scope and spirit of the following claims and equivalents thereto are claimed.

We claim:

1. An air scrubber/purifying filter comprising:
flocked media comprising:
an adhesively coated substrate;
a plurality of flock fibers flocked into the adhesively coated substrate; and
wherein the plurality of flock fibers comprises air disinfecting flock fibers.

2. The air scrubber/purifying filter of claim 1, wherein each of the plurality of flock fibers comprises at least one of:
a pass-through airborne pathogen destroying biocidal coating;
a pass-through airborne pathogen destroying virucidal coating;
a metalized coating;
a pass-over airborne pathogen destroying biocidal coating;
a pass-over airborne pathogen destroying virucidal coating;
a metal oxide coating; and
a metalized coating.

3. The air scrubbing/purifying filter of claim 1, wherein the adhesively coated substrate comprises a fabric substrate; and
wherein the air scrubbing/purifying filter further comprises a plurality of flocked fabric layers wherein the plurality of flocked fabric layers is arranged such that airflow through a filter cross-section passes through the plurality of flock fibers in a direction normal to an upright orientation of the plurality of flock fibers.

4. The air scrubbing/purifying filter of claim 3, wherein the plurality of flocked fabric layers comprises cross-flow flocked media (CFF) media.

5. The air scrubbing/purifying filter of claim 4, wherein the CFF media has a range of about 1 to 60 denier and a length of about 0.5 to about 10 mm.

6. The air scrubbing/purifying filter of claim 5, wherein CFF media comprises post fibrillated bi-component flock fiber having about 16-36 islands per sq. inch.

7. The air scrubbing/purifying filter of claim 1, wherein the adhesively coated substrate comprises a reticulated foam substrate; and
wherein the plurality of the air disinfecting flock fibers comprises germicide/biocide/virucide treated flock fibers.

8. The air scrubbing/purifying filter of claim 1, wherein the adhesively coated substrate comprises
a reticulated foam substrate; and
the plurality of flock fibers comprises a plurality of fibrillated bi-component fibers.

9. The air scrubbing/purifying filter of claim 8, wherein the plurality of fibrillated bi-component fibers comprises biocide/virucide treated fibrillated bi-component flock fibers.

10. The air scrubbing/purifying filter of claim 8, wherein the reticulated foam substrate comprises about 1 to 100 pores per sq. inch.

11. The air scrubbing/purifying filter of claim 8, wherein the reticulated foam substrate comprises an open cell reticulated foam.

12. The air scrubbing/purifying filter of claim 11, wherein the open cell reticulated foam comprises about 1-100 pores per sq. inch.

13. A method of making an air scrubber/purifying filter comprising:
providing a substrate;
providing flock fibers having disinfecting properties;
applying adhesive to the substrate; and
flocking the flock fibers having disinfecting properties onto the substrate to form a flocked fiber layer.

14. The method of claim 13, wherein the substrate is a reticulated foam substrate; and
and flocking the flock fibers comprises vacuum assisted flocking.

15. The method of claim 14, wherein the flock fibers comprise bi-component fibers and the method further comprises fibrillating the bi-component fibers.

16. The method of claim 15 further comprising
coating the flock fibers with at least one of:
a biocidal coating;
a virucidal coating; and
a metalized coating.
an air pass-over biocidal coating;
an air pass-over virucidal coating; and
a metalized coating.

17. The method of claim 13, wherein the air scrubber/purifying filter is a stacked assembly of Cross-Flow Flocked fabric filter layers; and
further comprises the steps of:
stacking a plurality of flocked fabric layers;
bonding the plurality of stacked, flocked fiber layers together on top of each other;
edgewise cross-cutting the bonded plurality of cross cut, stacked, flocked fiber layers; and
coating the flock fibers with one of:
an airborne pathogen destroying biocidal coating;
an airborne pathogen destroying virucidal;
a metalized coating;
a pathogen contaminated air pass-over biocidal coating;
a pathogen contaminated air pass-over virucidal coating;
a metalized coating; and
a metal oxide coating.

18. The method of claim 13, wherein the substrate comprises a porous reticulated foam (RF) substrate; and
the method further comprises applying a vacuum to the porous RF structure while flocking the flock fibers onto the porous RF substrate.
